(19) United States
(12) Patent Application Publication
(10) Pub. No.: US 2012/0283811 A1
(43) Pub. Date: Nov. 8, 2012

(54) BIODEGRADABLE, BIOABSORBABLE STENT ANCHORS

(75) Inventor: John Neilan, Co. Galway (IE)

(73) Assignee: Cook Medical Technologies LLC, Bloomington, IN (US)

(21) Appl. No.: 13/455,401

(22) Filed: Apr. 25, 2012

Related U.S. Application Data
(60) Provisional application No. 61/481,603, filed on May 2, 2011.

(51) Int. Cl. A61F 2/82; A61F 2/90

(52) U.S. Cl. 623/1.2; 623/1.15

(57) ABSTRACT

Biodegradable/bioabsorbable stent anchors are provided to prevent stent migration and permit repositioning and/or removal of the stent after the anchors or portion thereof has sufficiently degraded or been absorbed.

### RELATED APPLICATION

This application claims the benefit of priority from U.S. Provisional Application No. 61/481,603, filed May 2, 2011, and titled “Biodegradable, Bioabsorbable Stent Anchors”, the contents of which is incorporated herein by reference.

### TECHNICAL FIELD

The present invention relates to indwelling medical devices and more specifically, stents.

### BACKGROUND

Metal stents may be used to maintain a pathway within a bodily lumen. However, in many bodily areas, such as the esophageal tract, such stents are susceptible to migration from the area in which originally deployed. Such migration is generally undesired because the stent may damage surrounding tissue and may no longer maintain a pathway of the desired lumen.

However, the use of stent anchors to prevent stent migration is also problematic, as such anchors generally permit tissue in-growth into the stent, thereby preventing stent migration, but at the same time making removal and/or repositioning of the stent difficult or dangerous.

### BRIEF SUMMARY

In a first aspect, a stent is provided having a stent body including an elongated tubular body having a proximal portion, a distal portion, and a lumen extending throughout; wherein the elongated tubular body includes a substantially cylindrical configuration and is configured such that tissue in-growth is prevented; and an anchor disposed about the elongated tubular body of the stent, wherein the anchor includes a biodegradable or bioabsorbable material configured to degrade or absorb at about a known rate.

In a second aspect, a stent is provided having an elongated tubular body configured for maintaining open a bodily pathway; wherein the elongated tubular body includes a substantially cylindrical metal framework and a covering disposed on at least a portion of the substantially cylindrical metal framework; a plurality of anchors connected to the elongated tubular body, wherein the plurality of anchors include a biodegradable or bioabsorbable material configured to degrade or absorb at about a known rate; wherein the plurality of anchors further include: a first set of anchors including a first annular ring and a first plurality of struts connected to the first annular ring, wherein the first annular ring is connected to a proximal end of the elongated tubular body; and a second set of anchors including a second annular ring and a second plurality of struts connected to the second annular ring, wherein the second annular ring is connected to a distal end of the elongated tubular body, and wherein at least a portion of the struts each includes an open cell disposed therethrough.

### BRIEF DESCRIPTION OF THE SEVERAL VIEWS OF THE DRAWINGS

The embodiments will be further described in connection with the attached drawing figures. It is intended that the drawings included as a part of this specification be illustrative of the exemplary embodiments and should in no way be considered as a limitation on the scope of the invention. Indeed, the present disclosure specifically contemplates other embodiments not illustrated but intended to be included in the claims.

FIG. 1 illustrates a side view of an exemplary stent having biodegradable/bioabsorbable anchors; and

FIG. 2 illustrates a side view of an exemplary stent having biodegradable/bioabsorbable anchors.

### DETAILED DESCRIPTION OF PRESENTLY PREFERRED EMBODIMENTS

The exemplary embodiments illustrated herein provide exemplary apparatuses for providing non-permanent anchoring means for use with an indwelling medical device, such as a stent. The present invention is not limited to those embodiments described herein, but rather, the disclosure includes all equivalents and those intended to be included in the claims. Moreover, the embodiments illustrated herein can be used in any portion of the body benefiting from a removable or repositionable indwelling medical device, such as a stent, including but not limited to, the gastrointestinal region, esophageal region, duodenum region, biliary region, colonic region, as well as any other bodily region or field, and they are not limited to the sizes or shapes illustrated herein.

Throughout, patient is not limited to being a human being, indeed animals and others are contemplated. User is contemplated throughout the disclosure as being anyone or thing capable of using the device, including but not limited to, a human being and machine.

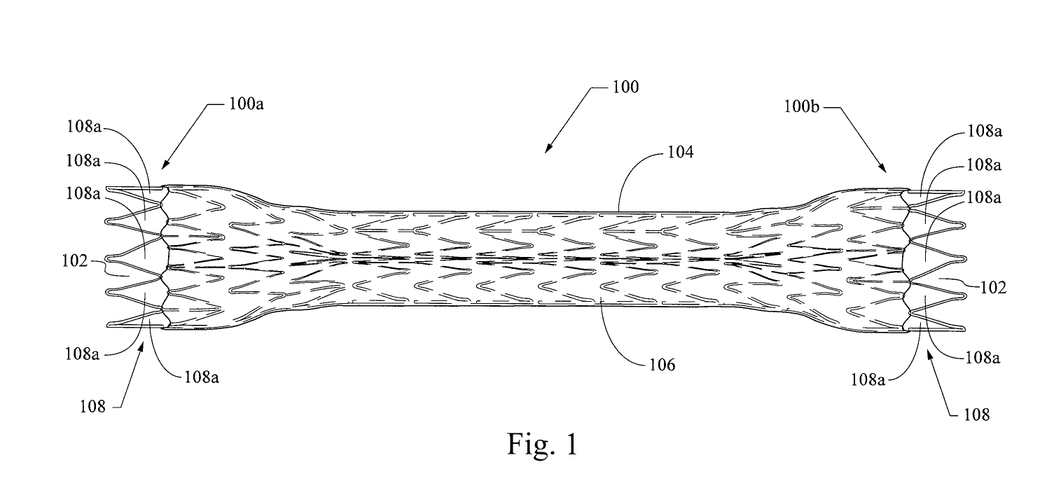

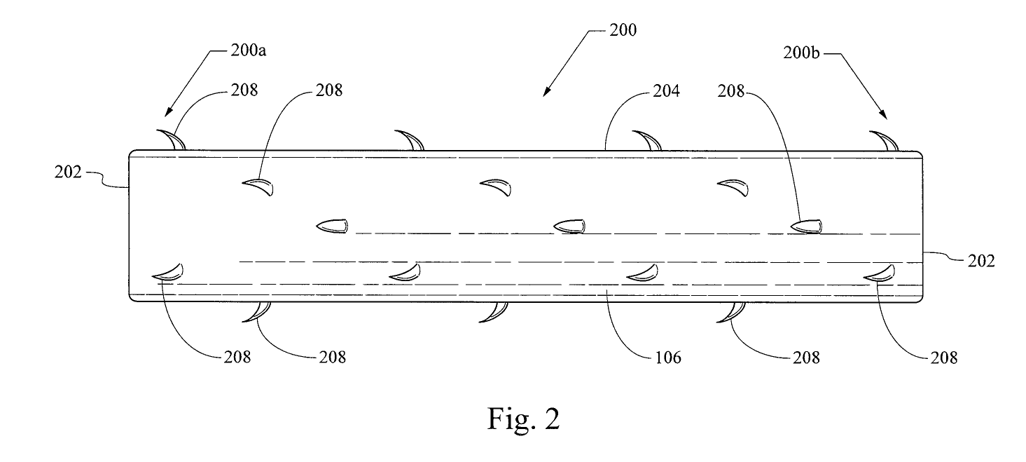

A more detailed description of the embodiments will now be given with reference to FIGS. 1-2. Throughout the disclosure, like reference numerals and letters refer to like elements. The present disclosure is not limited to the embodiments illustrated; to the contrary, the present disclosure specifically contemplates other embodiments not illustrated but intended to be included in the claims.

It has been discovered that providing biodegradable/bioabsorbable anchors to indwelling medical devices, such as a stent, including but not limited to, a metal stent, plastic stent, self-expanding sent, and other types of stents, allows the patient to benefit from a covered, coated, or sealed stent body (such as the use of a stent to open a bodily pathway while at the same time sealing a breach in the luminal wall) while at the same time reducing stent migration and permitting repositioning and removal of the stent after the anchors have sufficiently degraded or have been absorbed.

FIG. 1 illustrates a side view of exemplary stent 100 having biodegradable/bioabsorbable anchors 108. Stent 100 has an elongated tubular body having proximal portion 100a, distal portion 100b, and lumen 102 extending throughout. Illustrative stent 100 is a metallic stent having a substantially cylindrical metal framework wire mesh body 104, although other types and configurations of stents, and other indwelling medical devices, will benefit from the discovery, including but not limited to, plastic stents and self-expanding stents. Stent 100 is directed for use in the esophageal region, for example esophageal benign stenting, or, for example, to block off fistulas, although other uses and regions are contemplated, including but not limited to, the gastrointestinal region, duodenum region, biliary region, and colonic region.

Anchors 108 include an annular ring of struts/cells made from a biodegradable/bioabsorbable material, that further include open cell spaces 108a that permit tissue in-growth through, for example, an otherwise covered, coated, or sealed stent body 104 wherein tissue in-growth is generally not possible due to the covering, coating, material, or configuration from which stent body 104 includes. For example, as illustrated in FIG. 1, stent 100 includes a coating 106, such as a silicone membrane, although other materials are contemplated, to seal the exterior surface of stent body 104, thereby preventing, prohibiting, or limiting tissue in-growth. Open cell space 108a permits tissue in-growth such that stent 100 may be anchored into place to prevent, for example, migration. Other configurations are contemplated, including the use of more of less open cell spaces 108a, as well as, including but not limited to, anchors configured as barbs, as illustrated in FIG. 2.

Anchors 108 can be made from any biodegradable and/or bioabsorbable material, in whole or part, including but not limited to, those materials and techniques discussed in U.S. Patent App'l Publication No. 2005/0267560, filed May 23, 2005, entitled “Implantable Bioabsorbable Valve Support Frame,” incorporated herein by reference in its entirety. Preferably, the metallic bioabsorbable material is selected from a first group consisting of: magnesium, titanium, zirconium, niobium, tantalum, zinc and silicon. Also contemplated are mixtures and alloys of metallic bioabsorbable materials, including those selected from the first group. In some embodiments, the metallic bioabsorbable material can be an alloy of materials from the first group and a material selected from a second group consisting of: lithium, sodium, potassium, calcium, iron and manganese. Without being limited to theory, it is believed that the metallic bioabsorbable material from the first group may form a protective oxide coat upon exposure to blood or interstitial fluid. The material from the second group is preferably soluble in blood or interstitial fluid to promote the dissolution of an oxide coat. The bioabsorption rate, physical properties and surface structure of the metallic bioabsorbable material can be adjusted by altering the composition of the alloy. In addition, other metal or non-metal components, such as gold, may be added to alloys or mixtures of metallic bioabsorbable materials. Some preferred metallic bioabsorbable material alloy compositions include lithium-magnesium, sodium-magnesium, and zinctitanium, which can optionally further include gold. The frame itself, or any portion of the frame, can be made from one or more metallic bioabsorbable materials, and can further include one or more non-metallic bioabsorbable materials, as well as various non-bioabsorbable materials. The bioabsorbable material can be distributed throughout the entire frame, or any localized portion thereof, in various ways. In some embodiments, the frame can include a bioabsorbable material or a non-bioabsorbable material as a “core” material, which can be at least partially enclosed by other materials. The frame can also have multiple bioabsorbable materials stacked on all or part of the surface of a non-bioabsorbable core material. The frame can also include a surface area presenting both a bioabsorbable material and a non-bioabsorbable material.

A variety of bioabsorbable and biocompatible materials can be used to make medical device frames useful with particular embodiments disclosed herein, depending on the combination of properties desired. Properties such as flexibility, compliance, and rate of bioabsorption can be selected by choosing appropriate bioabsorbable materials. The properties of the bioabsorbable polymers may differ considerably depending on the nature and amounts of the comonomers, if any, employed and/or the polymerization procedures used in preparing the polymers.

Biodegradable polymers that can be used to form the support frame of a medical device, or can be coated on a frame, include a wide variety of materials. Examples of such materials, include but are not limited to, polyesters, polycarbonates, polyanhydrides, poly(amino acids), polyimines, polyphosphazenes and various naturally occurring biomolecular polymers, as well as co-polymers and derivatives thereof. Certain hydrogels, which are cross-linked polymers, can also be made to be biodegradable. These include, but are not necessarily limited to, polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, poly-alpha-hydroxy acids, trimethlyene carbonate, poly-beta-hydroxy acids, polyorganophosphazines, polyanhydrides, polyesteramides, polyethylene oxide, polyester-ethers, polyphosphoester, polyphosphoester urethane, cyanoacrylates, poly(trimethylene carbonate), poly(iminocarbonate), polyalkylene oxalates, polyvinylpyrolidone, polyvinyl alcohol, poly-N-(2-hydroxypropyl)-methacrylamide, polyglycols, aliphatic polyesters, poly(orthoesters), poly(ester-amides), polyanhydrides, modified polysaccharides and modified proteins. Some specific examples of bioabsorbable materials include poly(epsilon-caprolactone), poly(dimethyl glycolic acid), poly(hydroxyl butyrate), poly(p-dioxanone), polydioxanone, PEO/PLA, poly(lactide-co-glycolide), poly(hydroxybutyrate-covalerate), poly(glycolic acid-eo-trimethylene carbonate), poly(epsilon-caprolactone-co-p-dioxanone), poly-Lglutamic acid or poly-L-Iysine, polylactic acid, polylactide, polyglycolic acid, polyglycolide, poly(D,L-lactic acid), L-polylactic acid, poly(glycolic acid), polyhydroxyvalerate, cellulose, chitin, dextran, fibrin, casein, fibrinogen, starch, collagen, hyaluronic acid, hydroxyethyl starch, and gelatin.

It is contemplated that stents may benefit from more or fewer anchors, anchors having different configurations, and anchors disposed about a different portion of stent body 104 depending upon the needs of the patient and the area to be treated.

The types of biodegradable and/or bioabsorbable materials used, the amount of material used, and the construction of such material into different anchor shapes, sizes, numbers, and configurations, is contemplated such that anchors will dissolve or absorb into the body at a desired, known, or estimated rate of time.

For example, anchors 108 could be made from biodegradable or bioabsorbable materials, in whole or in part, having a construction such that anchors 108 will dissolve or be absorbed into the body in about three months' time, thus permitting the removal or repositioning of stent 100 after anchors 108 have sufficiently absorbed or degraded in whole or in part. Indeed, other constructions, configurations, and durations are contemplated depending upon the needs of the patient and the area to be treated.

It is contemplated that anchors 108, such as those illustrated in FIG. 1, are connected to stent body 104 using a variety of techniques, including but not limited to, having anchors 108 linked to stent body 104, integrally formed into stent body 104, or connected to stent body 104 after stent body 104 is constructed. Other methods for construction and techniques are contemplated depending upon the needs of the patient and the area to be treated.

Alternatively, the entirety of stent body 104 can be made from a biodegradable/bioabsorbable material. Such a configuration would provide, for example, for the treatment of fistulas without having to re-intervene to remove stent 100. Accordingly, a sealing coating 106, such as a the polymer jacket, over sent body 104, would pass through the digestive system once biodegradable/bioabsorbable stent body 104 had been absorbed/dissolved, thereby releasing it.

FIG. 2 illustrates a side view of exemplary stent 200 having biodegradable/bioabsorbable anchors 208 having a makeup similar to that which was illustrated in conjunction with FIG. 1. Stent 200 is a plastic stent having a substantially cylindrical stent body 204, although other types of stents, configurations of stents, and other indwelling medical devices are contemplated as are other materials. Stent 200 is an elongated tubular body having proximal portion 200a, distal portion 200b, and lumen 202 extending throughout. Disposed about stent body 204 are anchors 208, illustrated as barbs, which are biodegradable and/or bioabsorbable such that the migration of stent 200 is prevented or reduced, and stent 200 is able to be repositioned or removed after anchors 208 in whole or in part have sufficiently degraded or been absorbed. An optional coating 106 covers stent body 204. Other configurations are contemplated.

The principles described can be applied in whole or in part to any of the embodiments. For example, the principles illustrated in FIG. 1 can be applied in whole or in part to the embodiment and equivalents illustrated in FIG. 2 and visa versa.

From the foregoing, it can be seen that the present disclosure provides for indwelling medical devices, such as stents, having biodegradable/bioabsorbable anchors and coatings such that the stent is able to anchor into a bodily lumen and later be repositioned or removed after the anchors or portion thereof have sufficiently degraded or been absorbed.

What is claimed is:

1. A stent comprising:
a stent body comprising an elongated tubular body having a proximal portion, a distal portion, and a lumen extending throughout; wherein the elongated tubular body comprises a substantially cylindrical configuration and is configured such that tissue in-growth is prevented; and
an anchor disposed about the elongated tubular body of the stent, wherein the anchor comprises a biodegradable or bioabsorbable material configured to degrade or absorb at about a known rate.

2. The stent of claim 1, wherein the anchor further comprises a plurality of anchors.

3. The stent of claim 1, wherein the anchor comprises an annular ring comprising a plurality of struts, wherein at least a portion of the struts each comprises an open cell disposed therethrough.

4. The stent of claim 1, wherein the anchor comprises a barb.

5. The stent of claim 1, wherein the elongated tubular body comprises a wire configured into a mesh structure.

6. The stent of claim 1, wherein the elongated tubular body comprises a coating disposed about an outer surface of the elongated tubular body.

7. The stent of claim 1, wherein the anchor is disposed at a proximal or distal end of the elongated tubular body.

8. The stent of claim 1, wherein the elongated tubular body is configured to self-expand.

9. The stent of claim 1, wherein the anchor is configured to degrade or absorb in about a three-month time period.

10. A stent comprising:
an elongated tubular body configured for maintaining open a bodily pathway; wherein the elongated tubular body comprises a substantially cylindrical metal framework and a covering disposed on at least a portion of the substantially cylindrical metal framework;
a plurality of anchors connected to the elongated tubular body, wherein the plurality of anchors comprise a biodegradable or bioabsorbable material configured to degrade or absorb at about a known rate;
wherein the plurality of anchors further comprise:
a first set of anchors comprising a first annular ring and a first plurality of struts connected to the first annular ring, wherein the first annular ring is connected to a proximal end of the elongated tubular body; and
a second set of anchors comprising a second annular ring and a second plurality of struts connected to the second annular ring, wherein the second annular ring is connected to a distal end of the elongated tubular body, and
wherein at least a portion of the struts each comprises an open cell disposed therethrough.

11. The stent of claim 10, wherein the metal framework comprises a mesh structure.

12. The stent of claim 10, wherein the elongated tubular body is configured to self-expand.

13. The stent of claim 10, wherein the plurality of anchors are configured to degrade or absorb in about a three-month time period.
